(19) United States
(12) Patent Application Publication
(10) Pub. No.: US 2020/0163553 A1
(43) Pub. Date: May 28, 2020

(54) Label-Free Single and Multi-Photon Fluorescence Spectroscopy to Detect Brain Disorders and Diseases: Alzheimer, Parkinson, and Autism From Brain Tissue, Cells, Spinal Fluid, and Body Fluids

(71) Applicants: ROBERT ALFANO, New York, NY (US); LINGYAN SHI, Brooklyn, NY (US)

(72) Inventors: ROBERT ALFANO, New York, NY (US); LINGYAN SHI, Brooklyn, NY (US)

(21) Appl. No.: 16/773,533

(22) Filed: Jan. 27, 2020

Related U.S. Application Data
(63) Continuation of application No. 15/397,431, filed on Jan. 3, 2017.

(51) Int. Cl. A61B 5/00; G01N 33/483; G01N 21/64; A61B 10/00; A61B 10/02; G01N 33/68

(52) U.S. Cl. CPC A61B 5/0071; G01N 33/483; G01N 21/645; A61B 5/4076; G01N 21/6486; A61B 10/0045; A61B 2010/0077; G01N 33/6896; G01N 2800/2835; G01N 2800/2814; G01N 2021/6484; G01N 2800/2821; A61B 10/02

(57) ABSTRACT

A label free single or multi-photon optical excitation fluorescence spectroscopy for measuring the differences between the levels of fluorophores from tryptophan, collagen, reduced nicotinamide adenine dinucleotide (NADH), and flavins exist in brain samples from a of Alzheimer's disease (AD) and in normal (N) brain samples with label-free fluorescence spectroscopy. Relative quantities of these molecules are shown by the spectral profiles of the AD and N brain samples at excitation wavelengths and multi photons about 266 nm, 300 nm, 400 nm and 500 nm. The emission spectral profile levels of tryptophan and flavin were much higher in AD samples, while collagen emission levels were slightly lower and NADH levels were much lower in AD samples. These results yield a new optical method for detection of biochemical differences in animals and humans for Alzheimer's disease. These molecules in AD and N tissues and cells can be excited by 1PEF, 2PEF, 3PEF, 4 PEF using fs and ps pulses

### BACKGROUND OF THE INVENTION

### 1. Field of the Invention

The invention generally relates to diagnostic testing of brain disorders and diseases and, more specifically to label-free one or multiple photon-emission (“PE”) spectroscopy and imaging such as 1PE, 2PE and 3PE and 4 PE fluorescence (“PEF”) spectroscopy to detect brain disorders and diseases: Alzheimer, Parkinson and autism from brain tissue, cells, spinal fluid, and body fluids using ps and fs laser sources

### 2. Description of Prior Art

Alzheimer's disease (AD), a degenerative disorder that attacks neurons in the brain and leads to the loss of proper cognition, is the sixth leading cause of death in the United States, and from 2000-2010 the proportion of deaths resulting from AD in America has gone up 68%.[1] Although AD has been the focus of much scientific research in past years, there is still no cure or understanding of molecular mechanisms. A large proportion of people with AD remained undiagnosed; early diagnosis can help them make decisions for the future while they are still capable, and can allow people to receive early treatment to improve their cognition and increase the quality of their life as they live with AD.[2]

Physicians diagnose Alzheimer's disease with just an examination of a patient's state, inquiries into the familial history of psychiatric and neurological disorders, and a neurological exam.[1] Other newer methods of diagnosis include Magnetic Resonance Imaging (MRI) to look for Hippocampal atrophy,[3] Positron Emission Tomography (PET) scans, [4] and examining levels of beta-amyloid and tau protein in cerebrospinal fluids taken from the patient.[5]

Scientists continue to search for a better method to detect AD. Label-free optical spectroscopy offers a new tool to detect and understand the AD brain at the molecular level. Photonics offers a new and novel approach to give molecular information on AD. In 1984, Robert R. Alfano and his group of researchers at the City College of New York (C.C.N.Y.) pioneered the use of optical spectroscopy to detect cancer by looking at the native fluorescence levels of organic biomolecules.[6] This process of biomedical spectroscopy, using light and the native fluorescence of certain proteins and molecules within human tissue, has been expanded upon and applied to examine levels of tryptophan, reduced nicotinamide adenine dinucleotide NADH, flavin, and collagen in normal and cancerous breast tissue for diagnosing certain types of cancer. [7,8] The brain tissue is a smart tissue with different molecular components and structures in comparison to other body tissues. This past photonics work inspires the application of label free optical spectroscopy to the detection of AD and other brain disorders at molecular level in the brain.

Mitochondria play an essential role in energy production by oxidative phosphorylation and cell survival and death.[9,10] Mitochondrial dysfunction has been associated to a number of diseases including cancer and AD.[10-12] Early identification of mitochondrial dysfunction will be helpful for early detection and better understanding the mechanisms of AD. Intracellular coenzymes such as NADH and flavin adenine dinucleotide (FAD) play important roles in cellular oxidation-reduction (redox) reactions,[9] the can be potentially used as intrinsic biomarkers for detecting metabolic activities and mitochondrial dysfunction. Change of NADH-linked mitochondrial enzymes has been found in AD brain.[13, 14] Tryptophan kynurenine metabolism has also been reported involved in the pathogenesis of AD.[15]

Tryptophan, NADH, collagen, flavins and some other molecules have been examined as potential markers of Alzheimer's and neurological disease; Optical spectroscopy has not been employed to study the linear fluorescence of these biomarkers excited at various wavelengths in AD and normal (N) brain tissue The focus of this study is to apply optical fluorescence spectroscopy for 1PEF, 2PEF, 3PEF and 4PEF measuring fluorescence levels and imaging of key biomolecules (tryptophan, NADH, and collagen) in AD and N brain tissues using a mouse model of AD, and to propose a potential method for detection and diagnosis of Alzheimer's disease in humans. Different amounts of these label free biomolecules in brain are shown in FIGS. 1(a)-1(c) for different excitation wavelengths from 266 to 400 nm. These fluorescence spectral difference forms the teachings for the claims.

“Optical Biopsy” is a novel method using Raman and fluorescence spectroscopy at selected wavelengths to diagnose disease such as cancer, atherosclerosis, and brain disease without removing tissue from body, offering a new armamentarium. Key native molecules in tissues reveal the differences between diseased and normal tissues of various organs due to morphological and molecular changes in the tissue. The key label free optical methods are: fluorescence and Raman spectroscopies. Multiphoton has been used in brain research due to its deep tissue penetration capability and less photo-damage. Our group have applied two-photon microscopy for rodent brain tissue imaging, and found that the imaging depth and resolution were greatly increased.[9,10] Theoretical studies [11] demonstrated that applying three-photon microscopy would further improve the imaging depth and resolution. Various human tissue types (prostate, breast, lung, colon, arteries, and gastrointestinal) have been studied using optical biopsy. One can use lamps or LEDs to excite 1 PEF and femtosecond laser of types (Ti) and (Yb fiber and Er fiber and Supercontinuum) for 2 PEF, 3 PEF and 4PEF processes.

In the present study we measured the fluorescence spectroscopy in mouse brain tissue with an early stage of AD,[16] and in normal brain samples for comparison purpose. The objective is to develop a technique that applies biomolecules (tryptophan, NADH, and FAD) as intrinsic biomarkers for detecting early stage of AD in mouse brain tissue, and to propose a potential method for detection, diagnosis, and better understanding of AD in humans.

### SUMMARY OF THE INVENTION

We teach here the use of Linear and Nonlinear Optical Biopsy Spectroscopy to study brain and locate its disorders such as Alzheimer, Parkinson and Autism among others.

Optical spectroscopy has been considered a promising technique for cancer detection for more than two decades because of its advantages over the conventional diagnostic methods: no tissue removal, minimal invasiveness, less time consumption and reproducibility. Optical Biopsy was first used by Alfano et al., in 1984, who measured label free native fluorescence (NF), also called autofluorescence. Human tissue is mainly composed of an extracellular matrix of collagen fiber, proteins, fat, water, epithelial cells, which contains a number of key fingerprint native endogenous fluorophore molecules: tryptophan, collagen, elastin, reduced nicotinamide adenine dinucleotide (NADH), flavin adenine dinucleotide (FAD) and porphyrins. Tryptophan is an amino acid required by all forms of life for protein synthesis and other important metabolic functions, accounting for the majority of protein fluorescence. NADH and FAD are involved in the oxidation of fuel molecules and can be used to probe changes in cellular metabolism. It is well known that abnormalities in metabolic activity precede the onset of many diseases: carcinoma, diabetes, atherosclerosis, brain and Alzheimer's disease. The photonic tools use fiber spectroscopic ratiometer, fiber-optic endoscope for in vivo use for detecting in situ brain disorders pumped by linear and multiphoton excitation.

### BRIEF DESCRIPTION OF THE DRAWINGS

The above and other aspects, features and advantages of the present invention will be more apparent from the following description when taken in conjunction with the accompanying drawings, in which:

FIGS. 1(a)-1(c) show spectral profiles of AD and N brains at excitation wavelength (a) 266 nm, (b) 300 nm, and (c) 340 nm, respectively;

FIGS. 2(a) and 2(b) show absorption and fluorescence profiles of key biomolecules, respectively, FIG. 2(a) showing absorption of key molecules, and FIG. 2(b) showing emission of key molecules;

FIG. 3 shows the ratios of intensity peaks from three different regions of interest; FIGS. 4(a)-4(c) show the average of the first derivative of fluorescence profiles of AD and N brain tissues at excitation wavelength by 1PE or 2 or 3. or 4PE (a) 266 nm, (b) 300 nm, and (c) 340 nm;

FIG. 5(a)-5(h) show the two photon fluoresce images of (a) collagen, (b) NAHD, (c) Flavins and three photon fluorescence of (d) tryptophan in human breast cancer tissue and (e) collagen, (f) NADH, (g) Flavins and three photon fluorescence of (h) tryptophan in human normal breast tissue;

FIGS. 6(a) and 6(b) show fluorescence spectroscopy electronic states and elastic and Raman “vibrational states” for two-photon deeper tissue imaging;

FIGS. 7(a)-7(d) show native SHG, 2PEF, 3PEF and 4PEF Label Free (native molecules) emissions; and

FIG. 8 illustrates ICG absorption in relation to Soret peaks or bands.

### DETAILED DESCRIPTION

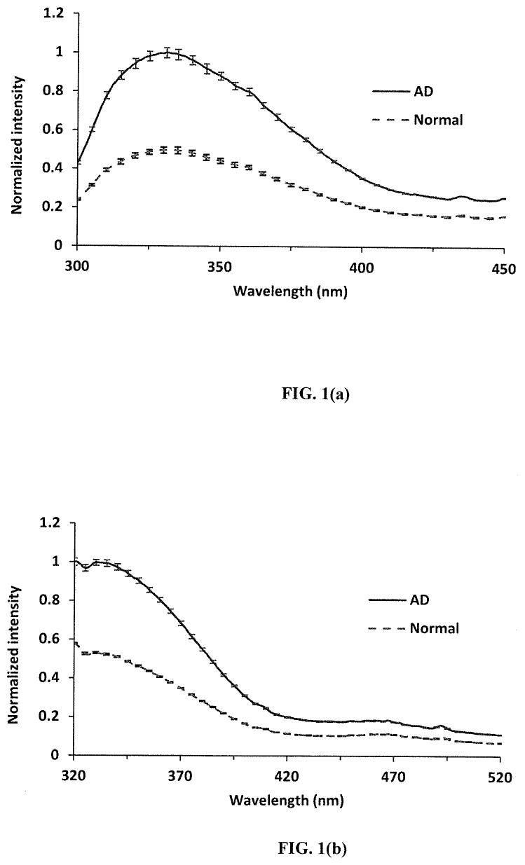

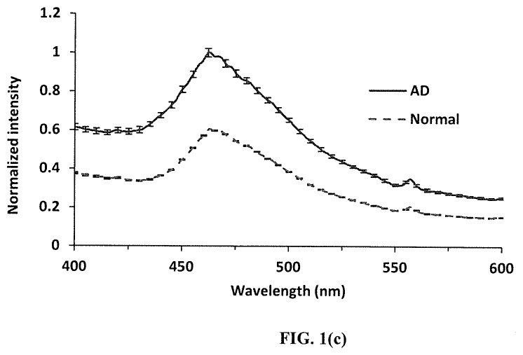

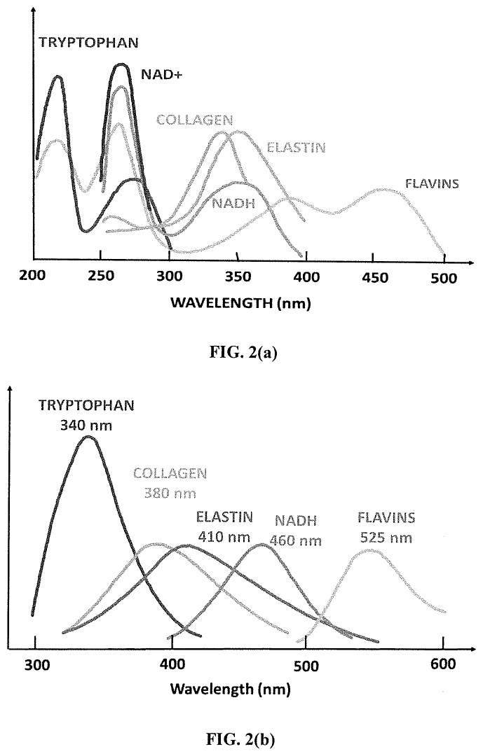

FIG. 1 displays the result of fluorescence spectral profiles in AD and N brain samples at excitation wavelengths 266 nm (FIG. 1a), 300 nm (FIGS. 1b), and 340 nm (FIG. 1c). Different excitation wavelengths were employed to determine the emission spectra of each biomolecule (tryptophan, NADH, and FAD), as shown in FIG. 2. Table 1 summarizes the emission wavelengths for assigned molecules at peak emissions in AD and N fresh brain tissues under different excitation wavelengths.

FIG. 1(a) shows that at excitation wavelength of 266 nm the fluorescence peaks of AD and N brain tissues are at the same wavelength (˜330 nm), corresponding to the wavelength of emission peaks of tryptophan (FIG. 2).[17] Significant difference of peaks of tryptophan was observed between AD and N brain (P=0.001). A weak secondary peak ranging from 430 to 460 nm is due to NADH, which may be caused by fluorescent resonance energy transfer from excited tryptophan to NADH and second singlet excitation from 266 nm. The averages of the two peak iintensities in AD brain are 2.01-fold (for tryptophan, 1.000 vs. 0.407) and 1.58-fold (for NADH, 0.268 vs. 0.169) higher, respectively, than those in N brain (Table 1).

FIG. 1(b) shows the scans at excitation wavelength of 300 nm, which are similar with emission spectra excited at 266 nm. The emission intensities of the AD and N brain tissues both peak in the range of 330-350 nm, which match the wavelength of the emission peak of tryptophan (FIG. 2). In addition, the weak second peaks are at 430-460 nm due to NADH. The mean peak intensity of tryptophan and NADH in AD brain tissue are 1.88-fold (1.000 vs. 0.532) and 1.61-fold (0.161 vs. 0.100) higher, respectively, than those in N brain tissue (Table 1).

FIG. 1(c) shows the scans at excitation wavelength of 340 nm. The emission peaks of AD and N in the range of 430-460 nm match the emission peak of NADH (FIG. 2), and the weak second peaks at 530-560 nm is due to FAD. The mean peak intensities of NADH and FAD are 1.65-fold (1.000 vs. 0.606) and 1.70-fold (0.352 vs. 0.207) higher, respectively, in AD brain compared to N brain (Table 1).

There are two comparable peaks in emission spectra of AD and N brain tissues in the ranges of 330-340 nm and 430-440 nm (FIG. 1c), which are consistent with the emission wavelengths of collagen and NADH, respectively (FIGS. 2(a) and 2(b)). The fluorescence peaks of collagen and NADH are 1.65-fold (1.000 vs. 0.606) and 1.70-fold (0.352 vs. 0.207) higher, respectively, in AD brain compared to N brain (Table 1).

Fluorescence spectroscopy measures allowed electronic transitions of various chromophores in the complex tissue structure. There are several natural label free fluorophores that exist in tissue and cells which, when excited with ultraviolet light, emit fluorescence in the ultraviolet and visible regions of the spectrum. Some of the absorption and emission spectra of these native endogenous fluorophore molecules are shown in FIGS. 2(a)-(b). The Flavins and NADH show changes in the spectra between their oxidized and reduced state. Single photon excitation fluorescence is applied to reveal the state of tissue and cells. The flavin and NADH show changes in the spectra between their oxidized and reduced state. When tryptophan, NADH and flavin are excited with ultraviolet light, they emit single photon excitation fluorescence in the ultraviolet and visible regions of the spectrum. The relatively large emission intensity from tissues and the need of broadly tunable excitation sources in the UV and visible has led researchers to develop lamp based fluorescence systems instead of lasers and now LEDs from 260 nm to 550 nm to excite the key biomolecules for 1 PEF. With the advent of femtosecond laser coupled with microscopy, these states can be excited by 1 PEF which is more a surface process and 2 PEF or 3 PEF or 4PEF for deeper penetration. The use of 2 PEF or 3 PEF or 4PEF will look into native fluorescence spectra from electronic states, to reveal the changes of these key metabolism related molecules.

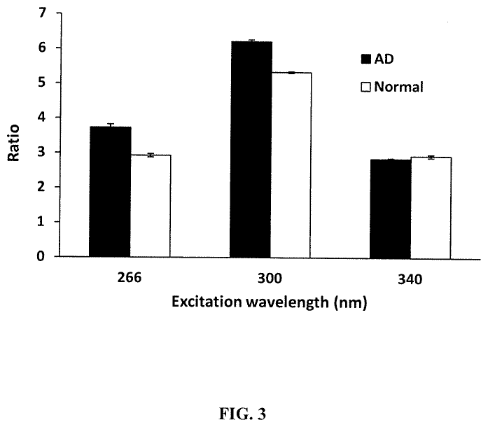

A basic fiber unit incorporates a fluorescence section and uses LEDs at 260 nm, 280 nm 300 nm, 350 nm, and 400 nm to excite Tryptophan, collagen, elastin, NADH, and FAD in brain disease. Femtosecond Ti lasers (700 nm to 1200 nm) can be used to excite the Key molecules (3 PEF for tryptophan @ 267 nm); and 2 PEF for collagen, NADH and flavins. See FIGS. 3(a)-5. Using Supercontinuum laser for excitation by 4PEF and 3PEF in 1200 nm to 2500 nm. Yb, Tm fiber with OPO, LIGO and LISO and CUNIYE lasers in SWIR region,

Significant differences of emission peaks were found in these molecules in AD and normal (N) brain. The fluorescence intensity levels from tryptophan: AD>N; from collagen: AD˜N; from NADH: N>AD and from flavin: AD>N. These observation provides effective techniques to explore an optical diagnosis of Alzheimer's disease by examining the spectral profiles of various molecules in brain tissue, eye fluid, body fluids, and/or spinal fluid ex vivo and in vivo using optical fibers.

An alternate way to differentiate the spectral profiles in AD or N brain is to compare the intensity ratio of tryptophan to NADH (Table 1, FIG. 3). The average values of the ratios are 3.73 in AD brain and 2.93 in N brain at the excitation wavelength of 266 nm, and 6.21 in AD and 5.33 in N excited at the wavelength of 300 nm. The reduced ration of tryptophan to NADH in AD indicates low efficiency of energy transfer from tryptophan (donor) to NADH (acceptor) which may be due to longer distance and fewer interactions between the two molecules. Comparing the spectral profiles (peaks) of tryptophan and NADH and their relative ration excited at the wavelength near absorption peak of tryptophan may be an applicable method for diagnosing AD. On the other hand, as shown in FIG. 3, the ratios of NADH to FAD in AD brain are not significantly different from the ratios in N brain, which indicates that analogous changes of NADH and FAD occurred in AD brain.

The first derivatives of emission spectra were calculated for comparing fluorescence properties of AD and N brain tissues. FIG. 4a-c show the mean profiles of the first derivative of emission spectra which were excited by monochromatic excitation lights of 266 nm, 300 nm, and 340 nm, respectively. At excitation wavelength of 266 nm, the ascending rate of emission intensity is higher in AD brain than that in N brain, the peaks of which are 0.039 vs. 0.0169; and the descending rate of intensity in AD is higher than that in N brain, the negative peaks of which are about −0.0159 vs. −0.0083. When excited at 300 nm, the maximum values of the ascending rate are 0.0078 in AD and 0.0034 in N brain; and the negative peaks of the descending rate are −0.0144 in AD and −0.0071 in N brain. However, the derivative of spectra from AD brain is close to that from N brain at excitation wavelength of 340 nm (FIG. 4c), due to the similar curve shapes of fluorescence spectra in both AD and N brain tissues (FIG. 1c). The derivative of spectral profiles could be used to measure instantaneous rate of change, the ration of the instantaneous change in the fluorescence intensity to that of its wavelength.

### Discussion

In our experimental results, fluorescence intensities of tryptophan, NADH, and FAD were higher in the brain tissues of a young transgenic AD mouse compared with N brain tissues. The increase in emission intensity at about 340 nm of direct pumping tryptophan shows more emission efficiency in AD than N, which may be due to decreased nonradiative Knr or increased Kr. This is because tryptophan may be in a cage and has fewer interactions to the host molecules in the environment in AD than in N brain. This observation is consistent with the results from THz research in AD and N. [18] Therefore, the vast disparity of tryptophan fluorescence levels in AD and N mouse brain scans proposes an important method for AD diagnosis. Mitochondrial abnormalities are correlated with AD, while intracellular NADH and FAD play important roles in mitochondrial dysfunction that allows them as potential biomarkers for diagnosis of AD,[9] and this is validated by the current study. Nevertheless, NADH-linked mitochondrial enzyme activity was reported to be down-regulated in AD patients,[13] our results showed higher NADH emission efficiency. One reason might be the different host environment of biological molecules in AD, in which NADH is farther from tryptophan and NADH itself may also have fewer interaction with the host environment. As a result, the emission intensity of NADH was higher in AD due to reduced nonradiative Knr or increased radiative Kr. Considering our objective was to detect AD in its early stage such that we used a young AD mouse, another reason may be due to overcompensation of NADH for dysfunction of energy metabolism in the early stage of AD. The future direction could use time resolved fluorescence which gives fluorescence rate (K_{f}=Kr+Knr) and combines with longer wavelength multiphoton excitation which offers deeper tissue penetration.

In the present study, the scattering of fluorescence intensity is small since 1) the emission is detected from <0.5 mm deep from the surface, and 2) the scattering coefficient and transport coefficient are smooth and flat, causing little or no influence on the measurements (as shown in FIG. 1).

In conclusion, the current study shows for the first time the fluorescence spectra of major molecular building blocks in brain of tryptophan, NADH, and FAD in AD and N mouse brain tissues. Fluorescence intensity levels of tryptophan, NADH, and FAD increased in AD brain tissues. This study verifies that tryptophan, NADH, and FAD can be employed as biomarkers for AD diagnosis. This work provides an effective technique to detect differences of fluorophore compositions in AD and normal brain tissues, and to diagnose AD by examining the spectral profiles of various fluorophores. This research can extend to employ ultrafast time resolved two photon excitation fluorescence spectroscopy for measuring the underlying relaxation times in AD.

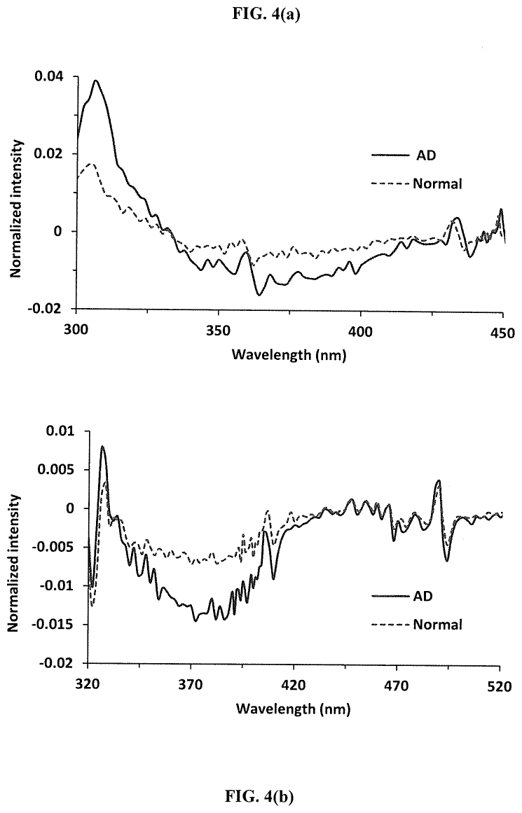

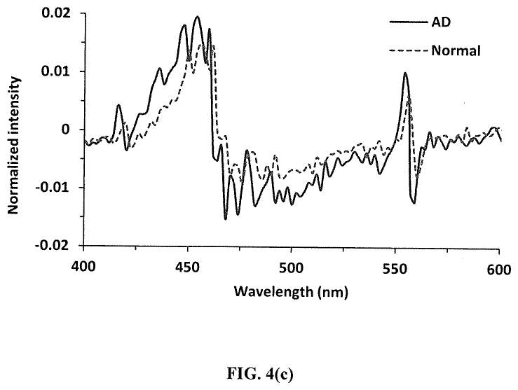

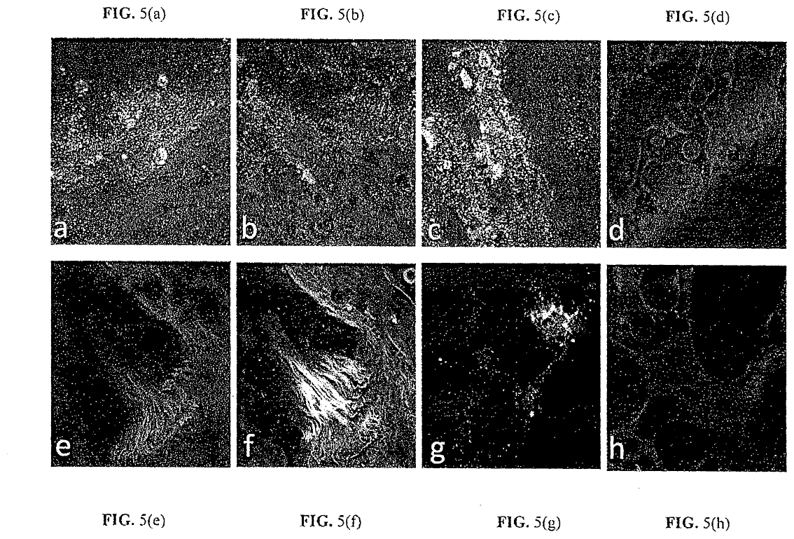

FIG. 5(a)-5(h) show the two photon fluoresce of (a) collagen, (b) NAHD, (c) Flavins and three photon fluorescence of (d) tryptophan in human breast cancer tissue and (e) collagen, (f) NADH, (g) Flavins and three photon fluorescence of (h) tryptophan in human normal breast tissue. A basic fiber unit incorporates a fluorescence section and uses LEDs at 260 nm, 280 nm 300 nm, 350 nm, and 400 nm to excite Tryptophan, collagen, elastin, NADH, and FAD in brain disease. Femtosecond Ti lasers (700 nm to 1200 nm) can be used to excite the Key molecules (3 PEF for tryptophan for 267 nm); and 2 PEF for collagen, NADH and flavins.

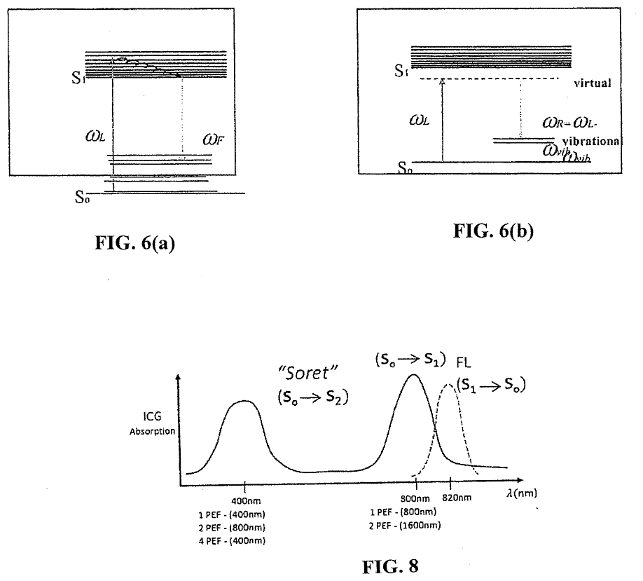

Referring to FIGS. 6(a) and 6(b) illustrates the fluorescence spectroscopy electronic states and elastic and Raman vibrational states for two-photon deeper tissue imaging.

Fluorescence spectroscopy measures allowed electronic transitions of various chromophores in the complex tissue structure. There are several natural label free fluorophores that exist in tissue and cells which, when excited with ultraviolet light, emit fluorescence in the ultraviolet and visible regions of the spectrum. Some of the absorption and emission spectra of these native endogenous fluorophore molecules are shown in FIGS. 2(a)-(b). The Flavins and NADH show changes in the spectra between their oxidized and reduced state. The relatively large emission intensity from tissues and the need of broadly tunable excitation sources in the UV and visible has led researchers to develop lamp based fluorescence systems instead of lasers and now LEDs from 260 nm to 550 nm to excite the key biomolecules for I PER These states can be excited by 1 PEF which is more a surface process and 2 PEF or 3 PEF for deeper penetration.

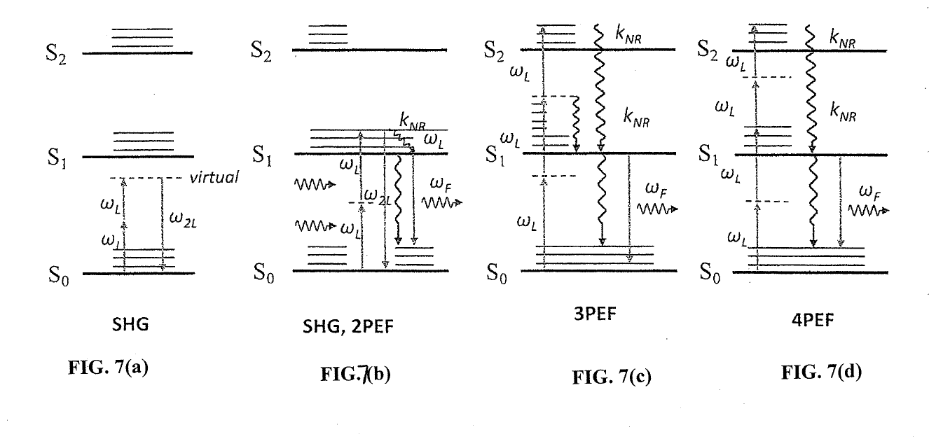

A basic fiber unit incorporates a fluorescence section and uses LEDs at 260 nm, 280 nm 300 nm, 350 nm, and 400 nm to excite Tryptophan, collagen, elastin, NADH, and FAD in brain disease. Femtosecond Ti lasers (700 nm to 1200 nm) can be used to excite the Key molecules (3 PEF for tryptophan a 267 nm); and 2 PEF for collagen, NADH and flavins. See FIGS. 6(a)-8.

Significant differences of emission peaks were found in these molecules in AD and normal (N) brain. The fluorescence intensity levels from tryptophan: AD>N; from collagen: ADN; from NADH: N>AD and from flavin: AD>N. These observation provides effective techniques to explore an optical diagnosis of Alzheimer's disease by examining the spectral profiles of various molecules in brain tissue, eye fluid, body fluids, and /or spinal fluid ex vivo and in vivo using optical fibers.

### Methods

### Animal Preparation

Mice were purchased from Jackson Laboratory and housed at the City College Animal Facility. A 3-month-old triple transgenic AD mice harboring PS1M146V, APPSwe and tauP301L transgenes in a uniform strain background [19] was used. Another N mouse at the same age was used as control. The experimental methods were in accordance with the guidelines and regulations approved by the Institutional Animal Care and Use Committee at the City College of the City University of New York. The protocol number is 841. The method used to prepare rodent brain tissue has been described in detail elsewhere.[18] A brief outline of the methods is given below with emphasis on the special features of the present experiments.

After anesthesia with a mixture of ketamine and xylazine (41.7 and 2.5 mg/kg body weight, respectively), the mouse was decapitated and the brain was dissected and taken out. Fresh brain tissue with the hippocampus region was quickly sliced coronally at thickness of ˜2 mm with a brain matrix (RWD Life Science Inc., Calif.). The fresh brain tissue slice was then immediately placed in a quartz cuvette. Regions of interest (ROI) in the hippocampus were measured 5 times at different spots in each AD and normal brain samples.

### Basic Theory of Fluorescence

It is well known that the fluorescence intensity I_{f} depends on efficiency Q from the radiative rate Kr and nonradiative rate Knr, where Q is given by [20]:

Q=Kr/(Kr+Knr) (1)

where Q equals to the ratio of numbers of photons emitted out to the numbers of photon pumped in (Nout/Nin). The intensity from excited molecules I_{f} is

I_{f}=(Ω/4π)(Q·n) (2)

where Ω is the solid angle and n is the number of excited molecules. Q value. The Knr depends on the interaction of molecules with their host environments. Weak interaction will lead to a small Knr and more emission intensity. When Knr»Kr, the emission is reduced.

Förster resonance energy transfer (FRET) is a mechanism for energy transfer between donor and acceptor via dipole-dipole coupling. Since the emission peak of tryptophan is around 340 nm and the absorption peak of NADH ranges from 340˜360 nm, energy transfer from excited donor (tryptophan) to acceptor (NADH) probably occurs in the biological tissues.[21] Effective donor to acceptor transfer can reduce emission from donor and enhance emission from acceptor. The transfer rate is

K_{DA}˜(1/τ_{D})(R₀/R)⁶ (3)

where R₀ is overlap between donor emission and acceptor absorption, τ_{D} is the fluorescence lifetime of donor, and R is the distance between donor and acceptor.

### FluoroMax-3 Fluorescence Spectrometer

The fluorescence of AD and N brain tissues was measured by a FluoroMax-3 fluorescence spectrometer (Horiba Jobin Yvon Inc.). A 150-W xenon lamp was used as the discharge light source in the spectrometer. There are two Czerny-Turner monochromators for excitation and emission respectively. The essential part of a monochromator is a reflection grating, which selects the wavelength being used. The gratings contain 1200 grooves mm⁻¹. A direct drive is used for each grating to scan the spectrum at up to 200 nm/s, the accuracy is better than 0.5 nm and repeatability is of 0.3 nm. The monochromatic excitation light strikes the sample, which is stored in a cuvette, and then emits fluorescence. The fluorescence light is directed into the emission monochromator, and is collected by the signal detector whose response ranges from 180-850 nm. Another detector named reference detector monitors the xenon lamp, and has good response from 190-980 nm.

The AD and N brain samples were excited at selected wavelengths 266 nm, 300 nm, and 340 nm, respectively, to examine the fluorescence peaks of each of tryptophan, NADH, and FAD. All measurements were performed by using a scanner (at 200 nm/sec), and the samples were held in cuvettes during the measurement.

Measurements of AD and N brain samples were each taken at three regions of interest, with the same spectral resolution of <1.0 nm (in bandpass unit) and integration time of 0.2 s at each excitation wavelength. Three groups of spectra were obtained at excitation 266 nm, 300 nm, and 340 nm, respectively. Each group contains three spectra from AD brain tissues and three from N brain. Average curve of these three spectra and maximum intensity were calculated. In each group, the spectral profiles were normalized to the maximum intensity of averaged spectra from AD brain. All averaged data was presented as mean±SD.

### Materials and Methods for Proof of Concept

### Animal Preparation

Mice were purchased from Jackson Laboratory and housed at the City College Animal Facility. A 3-month-old triple transgenic AD mouse harboring PS1M146V, APPSwe and tauP301L transgenes in a uniform strain background [12] was used. Another N mouse at the same age was used as control.

The mouse was anesthetized with a mixture of ketamine and xylazine (41.7/2.5 mg/kg body weight), then was decapitated and the brain was dissected. Fresh brain tissue with the hippocampus region was sliced coronally at a thickness of ˜2 mm, by using a brain matrix (RWD Life Science Inc, San Diego, Calif.). The fresh tissue slice was then immediately placed in a cuvette (Sigma-Aldrich, St. Louis, Mo.). Regions of interest (ROI) in the hippocampus were measured 5 times at different spots in each AD and normal brain samples

### Basic Theory of Fluorescence

It is well known that the fluorescence intensity I_{f} depends on efficiency Q from the radiative rate Kr and nonradiative rate Knr, the relationship can be written as [13]

Q=Kr/(Kr+Knr) (1)

Eq (1) for Q equals to the ratio of numbers of photons emitted out to the numbers of photon pumped in (Nout/Nin). The intensity from excited molecules I_{f} is

I_{f}=Ω/4π (Q·N) (2)

where Ω is the solid angle and N is the number of excited molecules. The Knr depends on the interaction of molecules with their host environments. Weak interaction will lead to a small Knr and give more emission intensity. When Knr»Kr the emission is reduced.

Förster resonance energy transfer (FRET) is a mechanism for energy transfer between donor and acceptor via dipole-dipole coupling. Since the emission peak of tryptophan is around 340 nm and the absorption peak of NADH ranges from 340˜360 nm, energy transfer from excited donor (tryptophan) to acceptor (NADH) probably occurs in the biological tissues.[14] Effective donor to acceptor transfer can reduce emission from donor and enhance emission from acceptor. The transfer rate is

K_{DA}˜(1/τ_{D})(R₀/R)⁶ (3)

where R₀ is overlap between donor emission and acceptor absorption, τ_{D} is the fluorescence lifetime of donor, and R is the distance between donor and acceptor.

### FluoroMax-3 Fluorescence Spectrometer

The fluorescence of Alzheimer and N brain tissues was measured by a FluoroMax-3 fluorescence spectrometer (Horiba Jobin Yvon Inc., Edison, N.J.). A 150-W xenon lamp was used as the discharge light source in the spectrometer. There are two Czerny-Turner monochromators for excitation and emission respectively. The essential part of a monochromator is a reflection grating, which selects the wavelength being used. The gratings contain 1200 grooves mm⁻¹. A direct drive is used for each grating to scan the spectrum at up to 200 nm/s, the accuracy is better than 0.5 nm and repeatability is of 0.3 nm. The monochromatic excitation light strikes the sample, which is stored in a cuvette, and then emits fluorescence. The fluorescence light is directed into the emission monochromator, and is collected by the signal detector whose response ranges from 180-850 nm. Another detector named reference detector monitors the xenon lamp, and has good response from 190-980 nm.

The AD and N brain samples were excited at wavelengths 266 nm, 300 nm, and 400 nm, to examine the fluorescence peaks of each of tryptophan, NADH, FAD, and collagen. All measurements were performed by using a scanner (at 200 nm/sec), and the samples were held in cuvettes during the measurement.

Measurements of AD and N brain samples were each taken at three regions of interest, with the same slit width of 2.0 nm (in bandpass unit) and integration time of 0.2 s at each excitation wavelength. Three groups of spectra were obtained at excitation 266 nm, 300 nm, and 340 nm, respectively. Each group contains three spectra from AD brain tissues and three from N brain. Average curve of the three spectra as well as its standard error of mean (SEM) and maximum intensity were calculated. In each group, the spectral profiles were normalized to the maximum intensity of averaged spectra from AD brain. All averaged data was presented as mean±SD.

### Results

One can use 1 PEF, 2 PEF and 3 PEF to excite the molecules in Table 1,

TABLE 1
Emission peaks in Alzheimer and N brain samples

| Excitation | | Normalized intensity | Normalized intensity | Ratio |
|---|---|---|---|---|
| wavelength | Tissue | of peak 1 | of peak 2 | (peak1/peak2) |
| | | tryptophan emission at 331 nm | NADH emission at 435 nm | |
| 266 nm | AD | 1.032 | 0.266 | 3.88 |
| | | 1.016 | 0.271 | 3.75 |
| | | 1.001 | 0.269 | 3.73 |
| | | 0.986 | 0.268 | 3.68 |
| | | 0.965 | 0.268 | 3.60 |
| | mean | 1.000 | 0.268 | 3.73 |
| | N | 0.522 | 0.174 | 3.01 |
| | | 0.495 | 0.170 | 2.91 |
| | | 0.495 | 0.169 | 2.93 |
| | | 0.491 | 0.167 | 2.93 |
| | | 0.480 | 0.167 | 2.88 |
| | mean | 0.497 | 0.169 | 2.93 |
| | | tryptophan emission at 335 nm | NADH emission at 492 nm | |
| 300 nm | AD | 1.013 | 0.164 | 6.19 |
| | | 1.014 | 0.161 | 6.29 |
| | | 1.005 | 0.161 | 6.25 |
| | | 0.989 | 0.161 | 6.15 |
| | | 0.979 | 0.158 | 6.19 |
| | mean | 1.000 | 0.161 | 6.21 |
| | N | 0.536 | 0.101 | 5.31 |
| | | 0.537 | 0.100 | 5.36 |
| | | 0.531 | 0.099 | 5.35 |
| | | 0.530 | 0.099 | 5.34 |
| | | 0.526 | 0.099 | 5.31 |
| | mean | 0.532 | 0.100 | 5.33 |
| | | NADH emission at 462 nm | FAD emission at 557 nm | |
| 340 nm | AD | 1.032 | 0.358 | 2.88 |
| | | 1.010 | 0.355 | 2.84 |
| | | 0.998 | 0.353 | 2.83 |
| | | 0.983 | 0.348 | 2.82 |
| | | 0.978 | 0.343 | 2.85 |
| | mean | 1.000 | 0.352 | 2.84 |
| | N | 0.606 | 0.212 | 2.86 |
| | | 0.609 | 0.206 | 2.95 |
| | | 0.609 | 0.205 | 2.97 |
| | | 0.605 | 0.206 | 2.93 |
| | | 0.602 | 0.207 | 2.92 |
| | mean | 0.606 | 0.207 | 2.928 |

AD: Alzheimer;
N: normal

FIG. 1 a shows that at excitation wavelength of 266 nm the fluorescence peaks of AD and N brain tissues are at the same wavelength (˜330 nm), corresponding to the wavelength of emission peaks of tryptophan (FIG. 2).[15] Significant difference of peaks of tryptophan was observed between AD and N brain (P=0.001). A weak secondary peak ranging from 430 to 460 nm is due to NADH, which is caused by fluorescent resonance energy transfer from excited tryptophan to NADH. The averages of the two peak intensities in AD brain are 2.01-fold (for tryptophan, 1.000 vs. 0.497) and 1.58-fold (for NADH, 0.268 vs. 0.169) higher, respectively, than those in N brain (Table 1).

FIG. 1(b) shows the scans at excitation wavelength of 300 nm, which are similar with emission spectra excited at 266 nm. The emission intensities of the AD and N brain tissues both peak in the range of 330-350 nm, which match the wavelength of the emission peak of tryptophan (FIG. 2). In addition, the weak second peaks are at 430-460 nm due to NADH. The mean peak intensity of tryptophan and NADH in AD brain tissue are 1.88-fold (1.000 vs. 0.532) and 1.61-fold (0.161 vs. 0.100) higher, respectively, than those in N brain tissue (Table 1).

There are two comparable peaks in emission spectra of AD and N brain tissues in the ranges of 330-340 nm and 430-440 nm (FIG. 1c), which are consistent with the emission wavelengths of collagen and NADH, respectively (FIG. 2). The fluorescence peaks of collagen and NADH are 1.65-fold (1.000 vs. 0.606) and 1.70-fold (0.352 vs. 0.207) higher, respectively, in AD brain compared to N brain (Table 1).

An alternate way to differentiate the spectral profiles in AD or N brain is to compare the intensity ratio of tryptophan to NADH (Table 1, FIG. 3a and b). Ratios of emission peaks at 331/435 nm when excited by the wavelength of 266 nm, 330/490 nm when excited by the wavelength of 300 nm, and 462/557 nm when excited by the wavelength of 340 nm. The average values of the ratio are 3.73 in AD brain and 2.93 in N brain at the excitation wavelength of 266 nm, and 6.21 in AD and 5.33 in N excited at the wavelength of 300 nm. The reduced ratio of tryptophan to NADH in AD indicates low efficiency of energy transfer from tryptophan (donor) to NADH (acceptor) which may be duo to longer distance (R) and fewer interactions between the two molecules. Comparing the spectral profiles (peaks) of tryptophan and NADH and their relative ratio excited at the wavelength near absorption peak of tryptophan may be an applicable method for diagnosing AD. On the other hand, as shown in FIG. 3c, the ratios of collagen to NADH in AD brain are not significantly different from the ratios in N brain, which indicates that analogous changes of collagen and NADH occurred in AD brain.

The first derivatives of emission spectra were calculated for comparing fluorescence properties of AD and N brain tissues. FIG. 4a-c show the mean profiles of the first derivative of emission spectra which were excited by monochromatic excitation lights of 266 nm, 300 nm, and 340 nm, respectively. At excitation wavelength of 266 nm, the ascending rate of emission intensity is higher in AD brain than that in N brain, the peaks of which are 0.039 vs. 0.0169; and the descending rate of intensity in AD is higher than that in N brain, the negative peaks of which are about −0.0159 vs. −0.0083. When excited at 300 nm, the maximum values of the ascending rate are 0.0078 in AD and 0.0034 in N brain; and the negative peaks of the descending rate are −0.0144 in AD and −0.0071 in N brain. However, the derivative of spectra from AD brain is close to that from N brain at excitation wavelength of 340 nm (FIG. 4c), due to the similar curve shapes of fluorescence spectra in both AD and N brain tissues (FIG. 1c). The derivative of spectral profiles could be used to measure instantaneous rate of change, the ratio of the instantaneous change in the fluorescence intensity to that of its wavelength.

In the experimental results, fluorescence intensities of tryptophan, collagen and NADH were higher in the brain tissues of a young transgenic AD mouse compared with N brain tissues. The increase in emission intensity at about 340 nm of direct pumping tryptophan shows more emission efficiency in AD than N, which may be due to decreased nonradiative Knr or increased Kr. This is because tryptophan may be in a cage and has fewer interactions to the host molecules in the environment in AD than in N brain. This observation is consistent with the results from THz research in AD and N. [16] Therefore, the vast disparity of tryptophan fluorescence levels in AD and N mouse brain scans proposes an important method for AD diagnosis. Increased intensity of collagen in AD mouse is consistent with others' finding that mouse neuronal expression of collagen increased, which could protect neurons against amyloid-β toxicity.[17] Besides, mitochondrial abnormalities always occur in AD brain. NADH-linked mitochondrial enzyme activity was reported to be down-regulated in AD patients.[18] However, our results showed higher NADH emission efficiency. One reason might be the different host environment of biological molecules in AD, in which NADH is farther from tryptophan and NADH itself may also have fewer interaction with the host environment. As a result, the mission intensity of NADH was higher in AD due to reduced nonradiative Knr or increased radiative Kr. Considering we used a young AD mouse, another reason may be due to overcompensation of NADH for dysfunction of energy metabolism in the early stage of AD. The future direction could use time resolved fluorescence which gives fluorescence rate (K_{f}=Kr+Knr) and combines with longer wavelength multiphoton excitation which offers deeper tissue penetration.

In the present study, the scattering of fluorescence intensity is small since 1) the emission is detected from <½ mm deep from the surface, and 2) the scattering coefficient and transport coefficient are smooth and flat, causing little or no influence on the measurements (as shown in FIG. 1).

This current study is the first teaching to investigate the fluorescence spectra of collagen, NADH, tryptophan, and flavin in Alzheimer and N mouse brain tissues. Fluorescence intensity levels of tryptophan, NADH, and collagen increased in AD brain tissues. This work provides effective techniques to detect differences of fluorophore compositions in AD and normal brain tissues, and to explore diagnosis of Alzheimer's disease by examining the spectral profiles of various fluorophores. This research can extend to employ ultrafast time resolved two photon excitation fluorescence spectroscopy for measuring the underlying relaxation times in AD.

FIG. 5(a)-5(h) show the two photon fluoresce of (a) collagen, (b) NAHD, (c) Flavins and three photon fluorescence of (d) tryptophan in human breast cancer tissue and (e) collagen, (f) NADH, (g) Flavins and three photon fluorescence of (h) tryptophan in human normal breast tissue. A basic fiber unit incorporates a fluorescence section and uses LEDs at 260 nm, 280 nm 300 nm, 350 nm, and 400 nm to excite Tryptophan, collagen, elastin, NADH, and FAD in brain disease. Femtosecond Ti lasers (700 nm to 1200 nm) can be used to excite the Key molecules (3 PEF for tryptophan for 267 nm); and 2 PEF for collagen, NADH and flavins.

### REFERENCES

1. Alzheimer's Association, “2013 Alzheimer's disease facts and figures,” Alzheimer's and Dementia 9 (2), 208-245 (2013).
2. Alzheimer's Disease International, “World Alzheimer Report 2011: The benefits of early diagnosis and intervention,” London (2011).
3. L. A. van de Pol, A. Hensel, W. M. van der Flier, P-J. Visser, Y. A. L. Pijnenburg, F. Barkhof, H. J. Gertz, and P. Scheltens, “Hippocampal atrophy on MRI in frontotemporal lobar degeneration and Alzheimer's disease,” J. Neurol. Neurosurg. Psychiatry 77 (4), 439-442 (2006).
4. G. E. Alexander, K. Chen, P. Pietrini, S. I. Rapoport, E. M. Reiman, “Longitudinal PET evaluation of cerebral metabolic decline in dementia: a potential outcome measure in Alzheimer's disease treatment studies,” The American J. of Psychology 159 (5), 738-745 (2002).
5. F. Hulstaert, K. Blennow, A. Ivanoiu, H. C. Schoonderwaldt, M. Riemenschneider, P. P. De Deyn, C. Bancher, P. Cras, J. Wiltfang, P. D. Mehta, K. Iqbal H. Pottel, E. Vanmechelen, and H. Vanderstichele, “Improved discrimination of AD patients using beta-amyloid(1-42) and tau levels in CSF,” Neurology 52 (8), 1555-1562, (1999).
6. R. R. Alfano, D. Tata, J. Cordero, P. Tomashefsky, F. Longo, and M. Alfano, “Laser induced fluorescence spectroscopy from native cancerous and normal tissue,” IEEE J. Quantum Electron. 20, 1507-1511 (1984).
7. Y. Pu, W. Wang, Y. Yang, and R. R. Alfano, “Native fluorescence of human cancerous and normal breast tissues analyzed with non-negative constraint methods,” Applied Optics 52 (6), 1293-1301 (2013).
8. L. A. Sordillo, P. P. Sordillo, Y. Budansky, Y. Pu, and R. R. Alfano, “Differences in fluorescence profiles from breast cancer tissues due to changes in relative tryptophan content via energy transfer: tryptophan content correlates with histologic grade and tumor size but not with lymph node metastases,” J. of Biomedical Optics 19 (12), 125002-1-125002-6 (2014).
9. L. Shi, L. Sordillo, R. R. Alfano, and A. Rodriguez-Contreras, “ Transmission in Near-Infrared Optical Windows for Deep Brain Imaging,” J. of Biophotonics 9 (1-2), 38-43, (2016).
10. L. Shi, A. Rodriguez-Contreras, Y. Budansky, Y. Pu, T. Nguyen, and R. R. Alfano, “Deep two-photon microscopic imaging through brain tissue using the second singlet state from fluorescent agent Chlorophyll α in spinach leaf,” J. of Biomedical Optics 19(6), 066009 (2014).
11. L. Shi, A. Rodriguez-Contreras and R. R. Alfano, “Gaussian beam in two-photon fluorescence imaging of rat brain microvessel,” J. of Biomedical Optics 19 (12), 126006 (2014).
12. S. Oddo, A. Caccamo, J. D. Shepherd, M. P. Murphy, T. E. Golde, R. Kayed, R. Metherate, M. P. Mattson, Y. Akbari, and F. M. Laferla,“Triple transgenic model of Alzheimer's disease with plaques and tangles: intracellular Ab and synaptic dysfunction,” Neuron 39, 409-421 (2003)
13. B. B. Das, F. Liu, and R. R. Alfano, “Time resolved Fluorescence and Photon Migration studies in Biomedical and model Random media,” Reports on Progress in Physics 60, 227 (1992).
14. T. Torikata, L. S. Forster, C. C. O'Neal Jr, and J. A. Rupley, “Lifetimes and NADH quenching of tryptophan fluorescence in pig heart lactate dehydrogenase,” Biochemistry, 18(2):385-390 (1979).
15. R. R. Alfano and Y. Pu, “Chapter 11—Optical Biopsy for Cancer Detection,” in H. Jelinkova (Eds.) [Laser for medical applications], Woodhead Publishing Limited, Cambridge, 325-367 (2013).
16. L. Shi, P. Shumyatsky, A. Rodriguez-Contreras, and R. R. Alfano, “Terahertz spectroscopy of brain tissue from a mouse model of Alzheimer's disease,” J. of Biomedical Optics 21(1), 015014-1—015014-5 (2016).
17. J. S. Cheng, D. B. Dubal, D. H. Kim, J. Legleiter, I. H. Cheng, G. Q. Yu, I. Tesseur, T. Wyss-Coray, P. Bonaldo, and L. Mucke, “Collagen VI protects neurons against Aβ toxicity,” Nature neuroscience, 12(2), pp.119-121 (2009).
18. C. B. Pocernich and D. A. Butterfield, “Acrolein inhibits NADH-linked mitochondrial enzyme activity: implications for Alzheimer's disease,” Neurotoxicity Research, 5(7), pp.515-519 (2003).

1. Method of detecting and imaging brain disorders and disease using an optical radiometer for detecting brain disorders and disease comprising the steps of:
(a) using a spectrometer optical analyzer at fixed wavelengths; a source for exciting a sample of molecules in cells and/or tissue within the range of 700 nm to 1200 nm ultrafast laser pulses (30 to 300 fs) by at least 2 PEF; and photo detectors for detecting fluorescence peaks of each of tryptophan, NADH, Flavins and collagen emitted from said molecules, said spectrometer optical analyzer including means for measuring the differences in the levels from native biomarkers of tryptophan, collagen, NADH and Flavin;
(b) collecting a sample of cells and/or tissue from a group consisting of brain tissue, eye fluid, body fluid and/or spinal fluid containing molecules found in a brain being examined AD and from a normal brain (N);
(c) exposing and exciting said molecules to selected wavelengths within the range of 200-800 nm by 1 PEF and/or by 700 nm to 1200 nm ultrafast laser pulses (30 to 300 fs) by 2 PEF, 3 PEF and/or 4PEF;
(d) detecting emission of fluorescence from the excited molecules; examining fluorescence peaks of each of tryptophan, NADH, flavins and collagen; comparing intensity levels of excitation and emission spectra for tryptophan, collagen, NADH and flavin; and
(e) establishing a diagnosis of Alzheimer's disease when the fluorescence intensity levels from a brain being examined (AD) and a normal brain (N) satisfy at least two of the following relationships:
Tryptophan level in AD is greater than tryptophan level in N
Collagen level in AD is approximately equal to collagen level in N
NADH level in N is greater than NADH level in AD
Flavin level in AD is greater than Flavin level in N.

2. A method as defined in claim 1, wherein exposure wavelengths cover the range of 260 nm to 500 nm.

3. A method as defined in claim 1, wherein exposure wavelengths cover the range of 320 nm to 550 nm.

4. A method as defined in claim 1, wherein optical fibers with photodetectors are used for detecting said optical peaks.

5. A method as defined in claim 4, wherein said photodetectors are selected from a group comprising CMOS, PMT and CCD.

6. A method as defined in claim 1, wherein spectral units are used to directly probe and excite different areas of the brain.

7. A method as defined in claim 6, wherein said spectral units are selected from a group comprising spectrograph, spectrometer and optical filters.

8. A method as defined in claim 1, wherein said source of excitation is selected from a group comprising xenon lamps, LEDs and femtosecond lasers for nonlinear 2 PEF and 3 PEF.

9. A method as defined in claim 8, further comprising a diffraction grating for intercepting the output of said source of excitation to provide desired excitation wavelengths for linear and non-linear 2 PEF and 3 PEF.

10. A method as defined in claim 1, further comprising an excitation monochromator arranged between said source of excitation and the sample for detecting and transmitting light within the range of 200-800 nm.

11. A method as defined in claim 1, further comprising and emission monochromator for detecting emissions from the sample within the range of 200-650 nm.

12. A method as defined in claim 1, wherein the sample is maintained in a cuvette.

13. A method as defined in claim 12, wherein excitation and emission monochromators are provided with said cuvette being positioned between said excitation and emission monochromators.
